(19) United States
(12) Patent Application Publication
(10) Pub. No.: US 2025/0122217 A1
(43) Pub. Date: Apr. 17, 2025

(54) ANHYDROUS CRYSTALLINE FORMS OF 2-((4S)-6-(4-CHLOROPHENYL)-1-METHYL-4H-BENZO[C]ISOXAZOLO[4,5-E]AZEPIN-4-YL)ACETAMIDE

(71) Applicant: CONSTELLATION PHARMACEUTICALS. INC., Boston, MA (US)

(72) Inventor: Louis Plamondon, Cambridge, MA (US)

(21) Appl. No.: 18/834,256

(22) PCT Filed: Jan. 31, 2023
(86) PCT No.: PCT/US2023/011919

§ 371 Date: Jul. 30, 2024

Related U.S. Application Data
(60) Provisional application No. 63/304,746, filed on Jan. 31, 2022.

(51) Int. Cl. C07D 498/04; A61K 31/55

(52) U.S. Cl. CPC C07D 498/04; A61K 31/55

(57) ABSTRACT

Provided is an anhydrous crystalline form of Pelabresib as well as its use for treating conditions associated with BET proteins.

### RELATED APPLICATIONS

This application claims the benefit of priority to U.S. Provisional Application No. 63/304,746, filed Jan. 31, 2022, the entire contents of which are incorporated by reference.

### BACKGROUND

Pelabresib, also known as CPI-0610, is a potent and selective small molecule designed to promote anti-tumor activity by selectively inhibiting the function of BET proteins to decrease the expression of abnormally expressed genes in cancer. Pelabresib has demonstrated a wide therapeutic window in lymphoma models, and its monohydrate form is currently being promoted in Phase 3 clinical trials for patients with myelofibrosis (MF) and related conditions. See e.g., NCT04603495. Given the therapeutic potential of Pelabresib, the development of stable crystalline forms of this drug remains an attractive area of investigation.

### SUMMARY

Provided herein is an anhydrous form of Pelabresib. Also provided are pharmaceutically acceptable compositions comprising an anhydrous form of Pelabresib, methods for its manufacture, and uses for treating conditions associated with the inhibition of BET proteins such as myelofibrosis.

### BRIEF DESCRIPTION OF THE FIGURES

FIG. 1 depicts an X-ray powder diffraction pattern (XRPD) for anhydrous crystalline Form B of Pelabresib.

FIG. 2 depicts a Differential scanning calorimetry (DSC) thermogram and thermal gravimetric analysis (TGA) overlay of B for anhydrous crystalline Form B of Pelabresib.

FIG. 3 shows the dynamic vapor sorption (DVS) profile for anhydrous crystalline Form B of Pelabresib.

### DETAILED DESCRIPTION

Provided is anhydrous crystalline Form B of Pelabresib.

The chemical name for Pelabresib is 2-((4S)-6-(4-chlorophenyl)-1-methyl-4H-benzo[c]isoxazolo[4,5-e]azepin-4-yl)acetamide. Pelabresib has a CAS number of 1380087-89-7, is disclosed as Compound 144 in U.S. Pat. No. 8,796,261, and has a chemical structure shown below.

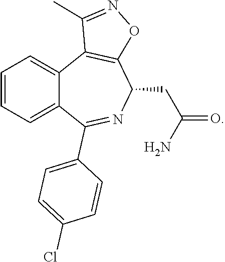 [SMILES: Cc1noc2c1-c1ccccc1C(c1ccc(Cl)cc1)=N[C@H]2CC(N)=O]

Monohydrate crystalline Form A of Pelabresib can be obtained following the disclosure in U.S. Pat. No. 9,969,747 and WO 2015/195863.

The term “anhydrous” means that the referenced crystalline form has substantially no water in the crystal lattice, e.g., less than 1% by weight as determined by Karl Fisher analysis.

“Anhydrous crystalline Form B” and “anhydrous crystalline Form B of Pelabresib” are used interchangeably.

As used herein, “crystalline” refers to a solid form of a compound wherein there exists long-range atomic order in the positions of the atoms. The crystalline nature of a solid can be confirmed, for example, by examination of the X-ray powder diffraction pattern.

Unless otherwise specified, anhydrous crystalline Form B is a single crystalline form. A “single crystalline form” means that the recited compound, i.e., Prelabresib, is present as a single crystal or a plurality of crystals in which each crystal has the same crystal form (e.g., anhydrous crystalline Form B). Percent by weight of anhydrous crystalline Form B is determined by the weight of anhydrous crystalline Form B divided by the sum weight of the anhydrous crystalline Form B, plus the weight of the other crystal forms present multiplied by 100%.

Chemical purity refers to extent by which the disclosed form is free from materials having different chemical structures. Chemical purity of the Prelabresib in the disclosed anhydrous crystalline Form B means the weight of Prelabresib divided by the sum of the weight of the Prelabresib plus materials/impurities having different chemical structures multiplied by 100%, i.e., percent by weight.

In one aspect, anhydrous crystalline Form B of Pelabresib is characterized by at least three X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by at least four X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by at least five X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by at least six X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by at least seven X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by at least eight X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by at least nine X-ray powder diffraction peaks at 2Θ angles selected from 11.56°, 11.86°, 14.61°, 17.91°, 20.46°, 21.01°, 24.06°, 24.16°, 24.76°, and 26.86°. In another aspect, anhydrous crystalline Form B of Pelabresib is characterized by X-ray powder diffraction peaks at 2Θ angles selected from 11.56°, 11.86°, 14.61°, 17.91°, 20.46°, 21.01°, 24.06°, 24.16°, 24.76°, and 26.86°.

In one aspect, anhydrous crystalline Form B of Pelabresib is characterized by X-ray powder diffraction peaks at 2Θ angles selected from 11.86°, 17.91°, and 21.01°. In another aspect, anhydrous crystalline Form B of Pelabresib is characterized by X-ray powder diffraction peaks at 2Θ angles selected from 11.86°, 17.91°, 21.01°, and 24.06°. In another aspect, anhydrous crystalline Form B of Pelabresib is characterized by X-ray powder diffraction peaks at 2Θ angles selected from 11.86°, 17.91°, 21.01°, 24.06°, and 24.16°. In another aspect, anhydrous crystalline Form B of Pelabresib is characterized by at least three X-ray powder diffraction peaks at 2Θ angles selected from 11.86°, 17.91°, 21.01°, 24.06°, and 24.16°. In another aspect, anhydrous crystalline Form B of Pelabresib is characterized by at least four X-ray powder diffraction peaks at 2Θ angles selected from 11.86°, 17.91°, 21.01°, 24.06°, and 24.16°.

In one aspect, anhydrous crystalline Form B of Pelabresib is characterized by an X-ray powder diffraction peak at 2Θ angle 11.86° and at least one additional X-ray powder diffraction peak at 2Θ angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least two additional X-ray powder diffraction peak at 2Θ angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least three additional X-ray powder diffraction peak at 2Θ angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least four additional X-ray powder diffraction peak at 20 angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least five additional X-ray powder diffraction peak at 2Θ angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least six additional X-ray powder diffraction peak at 2Θ angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least seven additional X-ray powder diffraction peak at 2Θ angle selected from 11.56°, 14.61°, 17.91°, 20.46°, 21.01°, 24.06°, 24.16°, 24.76°, and 26.86°. In another aspect, anhydrous crystalline Form B of Pelabresib is characterized by an X-ray powder diffraction peak at 2Θ angle 11.86° and at least eight additional X-ray powder diffraction peak at 20 angle selected from 11.56°, 14.61°, 17.91°, 20.46°, 21.01°, 24.06°, 24.16°, 24.76°, and 26.86°.

In one aspect, anhydrous crystalline Form B of Pelabresib is characterized by an X-ray powder diffraction peak at 2Θ angle 11.86° and at least one X-ray powder diffraction peak at 2Θ angle selected from 17.91°, 21.01°, 24.06°, and 24.16°. In another aspect, anhydrous crystalline Form B of Pelabresib is characterized by an X-ray powder diffraction peak at 2Θ angle 11.86° and at least two X-ray powder diffraction peak at 2Θ angle selected from 17.91°, 21.01°, 24.06°, and 24.16°. In another aspect, anhydrous crystalline Form B of Pelabresib is characterized by an X-ray powder diffraction peak at 2Θ angle 11.86° and at least three X-ray powder diffraction peak at 2Θ angle selected from 17.91°, 21.01°, 24.06°, and 24.16°.

In one aspect, anhydrous crystalline Form B of Pelabresib is characterized by one or more X-ray powder diffraction peaks at 2Θ angle in Table 2.

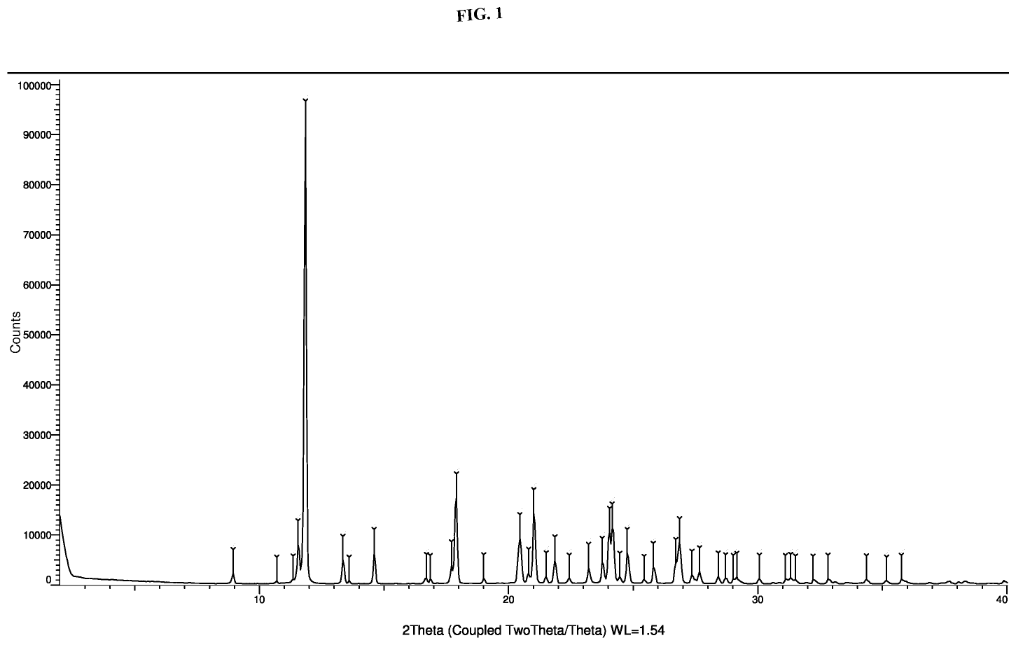

In one aspect, anhydrous crystalline Form B of Pelabresib is characterized by an XRPD substantially similar to FIG. 1.

In one aspect, anhydrous crystalline Form B is at least 60% a single crystalline form, at least 70% a single crystalline form, at least 80% a single crystalline form, at least 90% a single crystalline form, at least 95% a single crystalline form, or at least 99% a single crystalline form by weight.

In one aspect, anhydrous crystalline Form B has a chemical purity of at least 60%, at least 70%, at least 80%, at least 90%, at least 95%, or at least 99% by weight.

As used, “substantially the same XRPD pattern as shown in FIG. 1” means that for comparison purposes, the XRPD has at least 90%, at least 95%, or at least 99% of the peaks shown in FIG. 1. Alternatively, “substantially the same XRPD pattern as shown in FIG. 1” means that for comparison purposes, the XRPD is identical to FIG. 1, but for normal experimental variations such as e.g., temperature, processing conditions, instrumentation, and the like.

The term “amorphous” means a solid that is present in a non-crystalline state or form. Amorphous solids are disordered arrangements of molecules and therefore possess no distinguishable crystal lattice or unit cell and consequently have no definable long range ordering. Solid state ordering of solids may be determined by standard techniques known in the art, e.g., by X-ray powder diffraction (XRPD) or differential scanning calorimetry (DSC). Amorphous solids can also be differentiated from crystalline solids e.g., by birefringence using polarized light microscopy.

The 2-theta values of the X-ray powder diffraction patterns for Form B described herein may vary slightly from one instrument to another and also depending on variations in sample preparation and batch to batch variation due to factors such as temperature variation, sample displacement, and the presence or absence of an internal standard. Therefore, unless otherwise defined, the XRPD patterns/assignments recited herein are not to be construed as absolute and can vary ±0.2 degrees. In one aspect, variation for the angle 11.86° varies of ±0.15 degrees or less. It is well known in the art that this variability will account for the above factors without hindering the unequivocal identification of a crystal form. Unless otherwise specified, the 2-theta values provided herein were obtained using Cu Kα1 radiation.

Temperature values, e.g., for DSC peaks herein may vary slightly from one instrument to another and also depending on variations in sample preparation, batch to batch variation, and environmental factors. Therefore, unless otherwise defined, temperature values recited herein are not to be construed as absolute and can vary e.g., by ±5 degrees or ±2 degrees.

According to other aspects, the present disclosure relates to pharmaceutical compositions comprising anhydrous crystalline Form B and a pharmaceutically acceptable carrier.

The term “pharmaceutically acceptable carrier” refers to a non-toxic carrier, adjuvant, or vehicle that does not adversely affect the pharmacological activity of the compound with which it is formulated, and which is also safe for human use.

Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, dicalcium phosphate, potassium hydrogen phosphate, sodium chloride, zinc salts, colloidal silica, magnesium trisilicate, polyvinyl pyrrolidone, polyvinylpyrrolidone-vinyl acetate, cellulose-based substances (e.g., microcrystalline cellulose, hydroxypropyl methylcellulose, hydroxypropyl methylcellulose acetate succinate, hydroxypropyl methylcellulose Phthalate), starch, lactose monohydrate, mannitol, sodium lauryl sulfate, and crosscarmellose sodium, polyethylene glycol, sodium carboxymethylcellulose, polyacrylates, polymethacrylate, waxes, polyethylene-polyoxypropylene-block polymers, polyethylene glycol and wool fat.

Diseases and conditions treatable according to the methods described herein include, but are not limited to, cancer and other proliferative disorders, inflammatory diseases, sepsis, autoimmune disease, and viral infection. Thus, one aspect is a method of treating a subject having a disease, disorder, or symptom thereof the method including administration of anhydrous crystalline Form B or composition herein to the subject.

The present disclosure further relates to a method for treating cancer or another proliferative disorder by administration of an effective amount of anhydrous crystalline Form B as described herein to a mammal, in particular a human in need of such treatment. In some aspects, the disease to be treated is cancer. Examples of cancers treated include, but are not limited to, adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In one aspect, the present disclosure further relates to a method for treating myelofibrosis comprising administering to a subject in need thereof, an effective amount of anhydrous crystalline Form B.

In one aspect, the present disclosure further relates to a method for treating essential thrombocythemia (ET) comprising administering to a subject in need thereof, an effective amount of anhydrous crystalline Form B.

In some aspects, the present disclosure provides a method of treating a benign proliferative disorders, infectious and noninfectious inflammatory events and autoimmune and other inflammatory diseases, systemic inflammatory response syndromes, and viral infections.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided crystalline form in the composition will also depend upon the particular compound in the composition.

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence.

The term “effective amount” or “therapeutically effective amount” includes an amount of a compound described herein that will elicit a biological or medical response of a subject, e.g., a dosage of between 0.001-100 mg/kg body weight/day of anhydrous Form B.

### EXEMPLIFICATION

Anhydrous crystalline Form B can be prepared according to the following procedures outlined below in Table 1. The starting material, amorphous Pelabresib, was prepared following the procedures outlined for compound 144 in U.S. Pat. No. 8,796,261.

TABLE 1

| Solvent | Experimental Conditions |
|---|---|
| isopropanol | Partial slow evaporation, transferred to refrigerator, solids observed |
| | approximately 4 days later |
| ethanol | Partial slow evaporation, transferred to refrigerator, solids observed |
| | approximately 4 days later |
| ethanol and water | Vapor diffusion, solids observed approximately 4 days later |
| ethanol | Scale-up of Form B; dissolved approximately 1 g of Form A in 5 mL |
| | EtOH, filtered through 0.2 μm filter, stirred at RT for approximately |
| | 5 hrs |
| ethanol | Repeat of scale-up, dissolved approximately 1 g of Form A in 5 mL |
| | EtOH, filtered through a 0.2 μm filter, stirred at RT for 1 day |
| heptane:ethanol | low cooling at approximately 70° C.; solution became clear after |
| (70:30) | adding 5 mL to approximately 214 mg of starting material, filtered, |
| | slow cooled to RT with stirring, isolated 1 day later |
| heptane:ethanol | Slow cooling at approximately 70° C.; solution became clear after 2 |
| (60:40) | mL in 337 mg of starting material but ppt observed quickly at |
| | elevated temperature, filtered, slow cooled to RT with stirring, |
| | isolated 1 day later |
| heptane:ethanol | Slurry at RT for approximately 5 days |
| (95:5) | |
| heptane:ethanol | Slurry at RT for approximately 5 days |
| (90:10) | |
| ethyl | Slow cooling at approximately 55° C., let stir at RT for approximately |
| acetate:heptane | 1 day, insufficient solids, added 5 mL heptane, let stir at RT for |
| (2:1) | approximately 1 day, yield 50% |
| methyl tert butyl | Slow cooling at approximately 55° C., let stir at RT for approximately |
| ether:heptance | 1 day, insufficient solids, added 5 mL heptane, let stir at RT for |
| (10:3) | approximately 1 day |
| ethyl | Dissolved in 1 mL EtOAc, no precipitation after approximately 1 |
| acetate/heptane | day, added 3 mL heptane, oily precipitation, let stir at RT for |
| | approximately 1 day |
| methyl tert butyl | Slurry at RT for approximately 2 days |
| ether | |
| tetrahydrofuran:heptane | Slow cooling at approximately 60° C., solution became clear after 4 |
| (40:60) | mL to 275 mg (black gummy spots noted on bottom and edge of |
| | vial), filtered, slow cool to RT with stirring, precipitation noted |
| | immediately, let stir at RT for approximately 1 day |

The XRPD spectrum for Form B is shown in FIG. 1. Representative data points are shown in Table 2.

TABLE 1

| Angle | d Value | Rel. Intensity |
|---|---|---|
| 8.95 | 9.87 | 2.3% |
| 10.7 | 8.26 | 0.7% |
| 11.36 | 7.79 | 0.9% |
| 11.56 | 7.65 | 8.6% |
| 11.86 | 7.46 | 100.0% |
| 13.36 | 6.62 | 5.2% |
| 13.61 | 6.50 | 0.7% |
| 14.61 | 6.06 | 6.7% |
| 16.71 | 5.30 | 1.3% |
| 16.86 | 5.26 | 1.0% |
| 17.71 | 5.00 | 4.0% |
| 17.91 | 4.95 | 18.8% |
| 19.01 | 4.67 | 1.2% |
| 20.46 | 4.34 | 9.9% |
| 20.81 | 4.27 | 2.3% |
| 21.01 | 4.23 | 15.4% |
| 21.51 | 4.13 | 1.6% |
| 21.86 | 4.06 | 5.1% |
| 22.44 | 3.96 | 1.1% |
| 23.21 | 3.83 | 3.4% |
| 23.76 | 3.74 | 4.7% |
| 24.06 | 3.70 | 11.3% |
| 24.16 | 3.68 | 12.2% |
| 24.46 | 3.64 | 1.5% |
| 24.76 | 3.59 | 6.7% |
| 25.44 | 3.50 | 0.8% |
| 25.81 | 3.45 | 3.6% |
| 26.71 | 3.33 | 4.5% |
| 26.86 | 3.32 | 9.0% |
| 27.36 | 3.26 | 1.9% |
| 27.66 | 3.22 | 2.7% |
| 28.41 | 3.14 | 1.5% |
| 28.71 | 3.11 | 1.2% |
| 29.02 | 3.07 | 1.0% |
| 29.16 | 3.06 | 1.5% |
| 30.07 | 2.97 | 1.1% |
| 31.11 | 2.87 | 1.0% |
| 31.31 | 2.85 | 1.3% |
| 31.51 | 2.84 | 0.9% |
| 32.22 | 2.78 | 0.9% |
| 32.81 | 2.73 | 1.1% |
| 34.36 | 2.61 | 1.0% |
| 35.17 | 2.55 | 0.7% |
| 35.76 | 2.51 | 1.1% |

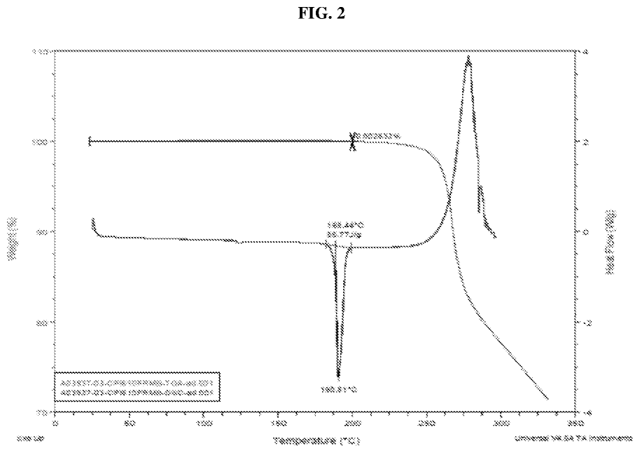

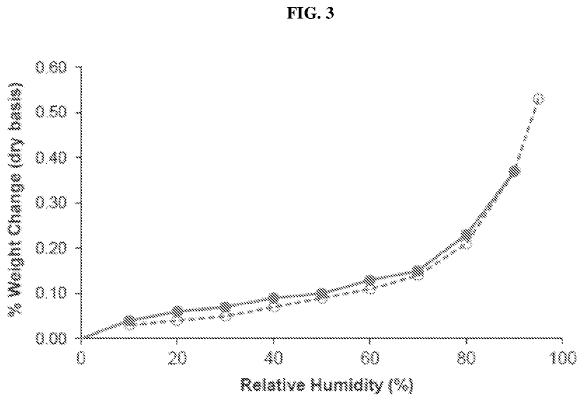

Thermal data shows Form B is anhydrous and non-solvated with a single sharp endotherm onset at about 188° C. See FIG. 2. The DVS data indicate that Form B is non-hygroscopic with about 0.5% weight gain at 95% RH. See FIG. 3.

### Competitive Slurry Experiments

The following competitive slurry experiments were performed using inventive anhydrous Form B and monohydrate Form A disclosed in U.S. Pat. No. 9,969,747.

Aqueous mixtures were created and tested in duplicate for water activity at ambient temperature. Amorphous Pelabresib was used to create a saturated solution in the respective solvent. The supernatant was added to solids of monohydrate Form A and anhydrous Form B and allowed to stir at the respective temperature for about 5-7 days. In all samples except for the butanol:water mixture, a small quantity of Form C was also added to the competitive slurry. Form C can be prepared by slurring amorphous pelabresib in water:ethanol (90:10) for about 5 days The results of the competitive slurries are shown below in Table 3.

TABLE 3

| | Water | | |
|---|---|---|---|
| Solvent | Activity | Temperature | XRD result |
| IPA:water | 0.27 | RT | Form B |
| (98:2) | | 40° C. | Form B + small peak |
| EtOH:water | 0.54 | RT | Form B |
| (90:10) | | 40° C. | Form B |
| THF:water | 0.97 | RT | Form C |
| (10:90) | | 40° C. | Form A |
| 2-butanol:water | 0.72 | RT | Form B |
| (95:5) | | 40° C. | Form B |

The results of the competitive slurries indicate that Form B is thermodynamically stable at water activities at or below 72%, and Form A is thermodynamically stable at water activities at or above 97%.

### Pharmacokinetics of Anhydrous Form B in Beagle Dogs

Single dose PK parameters in plasma were determined following oral gavage administration of micronized anhydrous Form B at 5 mg/kg to male beagle dogs (n=6/Group) in 0.5% (w/v) aqueous Methocel (4000 cps). The dose volume of PO groups was 2 mL/kg. For intravenous (0.5 mg/kg) and oral gavage (5 mg/kg), plasma samples were collected at the following time points: 0, 0.25, 0.5, 1, 2, 4, 6, 8, 12, 24 h post dose and analyzed. Following oral administration (5 mg/kg, 2.5 mg/mL, suspension), Tmax was 1.17±0.41 hr, Cmax was 1747±225 ng/mL and AUC0-24 hr was 12133±1452 h*ng/mL. (5 mg/kg, 2.5 mg/mL, suspension).

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. Anhydrous crystalline Form B of 2-((4S)-6-(4-chlorophenyl)-1-methyl-4H-benzo[c]isoxazolo[4,5-e]azepin-4-yl)acetamide.

2. The anhydrous crystalline Form B of claim 1, characterized by at least three X-ray powder diffraction peaks at 2Θ angles selected from 11.56°, 11.86°, 14.61°, 17.91°, 20.46°, 21.01°, 24.06°, 24.16°, 24.76°, and 26.86°.

3. The anhydrous crystalline Form B of claim 1, characterized by at least four X-ray powder diffraction peaks at 2Θ angles selected from 11.56°, 11.86°, 14.61°, 17.91°, 20.46°, 21.01°, 24.06°, 24.16°, 24.76°, and 26.86°.

4. The anhydrous crystalline Form B of claim 1, characterized by at least five X-ray powder diffraction peaks at 2Θ angles selected from 11.56°, 11.86°, 14.61°, 17.91°, 20.46°, 21.01°, 24.06°, 24.16°, 24.76°, and 26.86°.

5. The anhydrous crystalline Form B of claim 1, characterized by at least six X-ray powder diffraction peaks at 2Θ angles selected from 11.56°, 11.86°, 14.61°, 17.91°, 20.46°, 21.01°, 24.06°, 24.16°, 24.76°, and 26.86°.

6. The anhydrous crystalline Form B of claim 1, wherein the crystalline Form 1 is at least 90% a single crystalline form by weight.

7. The anhydrous crystalline Form B of claim 1, wherein the crystalline Form 1 is at least 95% a single crystalline form by weight.

8. The anhydrous crystalline Form B of claim 1, wherein the compound has a chemical purity of at least 90% by weight.

9. The anhydrous crystalline Form B of claim 1, wherein the compound has a chemical purity of at least 95% by weight.

10. The anhydrous crystalline Form B of claim 1, wherein the compound has a chemical purity of at least 99% by weight.

11. A pharmaceutical composition comprising the anhydrous crystalline Form B of claim 1, and a pharmaceutically acceptable carrier.

12. A method of treating a disease or disorder associated with the inhibition of BET in a subject in need thereof comprising administering to the subject an effective amount of the anhydrous crystalline Form B of claim 1.

13. The method of claim 12, wherein the disease or disorder is myelofibrosis.
